(19) United States
(12) Patent Application Publication
(10) Pub. No.: US 2025/0049297 A1
(43) Pub. Date: Feb. 13, 2025

(54) Endoscope and endoscope system for cardio-float applications

(71) Applicant: NET New Electronic Technology GmbH, Finning (DE)

(72) Inventors: Peter PUSCH, Gröbenzell (DE); Uwe-Heinz POST, Fürstenfeldbruck (DE); Thomas Karl DÄUBLER, Mannheim (DE)

(21) Appl. No.: 18/717,500

(22) PCT Filed: Dec. 9, 2022
(86) PCT No.: PCT/EP2022/085182

§ 371 Date: Jun. 7, 2024

(30) Foreign Application Priority Data
Dec. 9, 2021 (DE) 102021132567.6

(51) Int. Cl. A61B 1/00; A61B 1/05; A61B 1/06

(52) U.S. Cl. CPC A61B 1/00071; A61B 1/00135; A61B 1/05; A61B 1/0669

(57) ABSTRACT

An endoscope for cardio-float applications comprises an endoscope head and a flexible sheath. A camera with a distal viewing direction is arranged in the endoscope head. The endoscope head has a shielding, earth-related, electrically conductive protective sheath, within which an image sensor and a camera driver are arranged. The endoscope head also has insulation that completely encloses at least the protective sheath and has a breakdown voltage of at least 1 kV.

The invention relates to an endoscope and an endoscope system for cardio-float applications.

An endoscope system comprises both the endoscope itself and an external catheter, as well as display and control devices for these.

In surgery, endoscopes are an indispensable tool for understanding processes inside the human or animal body. Imaging procedures have long been known in which a camera is used to record conditions in the human or animal body. For example, images are commonly taken during a colonoscopy.

However, not every endoscope is equally suitable for every area of the body. For example, an endoscope for a colonoscopy is not suitable for heart surgery.

Rigid endoscopes are known, e.g. from US 2020/0 113 429 A1 and US 2014/0 155 758 A1, which have a camera at the proximal end. These endoscopes are characterized by a short rigid path from the proximal endoscope tip to the distal end. They have no or only a predetermined curvature. These endoscopes are not suitable for insertion with an elastic catheter into a curved patient cavity.

Flexible endoscopes are also known in which image conductors guide images from the distal to the proximal end, at the end of which there is an image sensor. The disadvantage here is the low resolution that can be achieved by the system. Glass image guides are also often brittle and can break easily. They only have a certain degree of flexibility.

A rigid endoscope with several joints is known from U.S. Pat. No. 11,039,738, whereby a camera is formed at the proximal end.

DE 10 2016 099 476 B3 discloses an endoscopy device that has a rigid endoscope shaft. The endoscope is characterized by the fact that a galvanic barrier is set up between the electronic shielding and the endoscope shaft, via which the endoscope shaft is capacitively coupled to the electronic shielding.

JP 3233488 B2 discloses an endoscope having an outer electrical shield arranged on the outer periphery of the endoscope. The endoscope has a plurality of insulating materials with different dielectric constants which are concentrically stacked.

The purpose of the present invention is to provide an endoscope in which areas of the human body that are very sensitive to external electrical currents can be optically detected in a living state (in situ) with a high resolution.

A further task of the invention is to provide an endoscope which is flexible in order to reach inaccessible areas of the human or animal body.

A further task of the invention is to provide an endoscope which is very thin in order to reach into narrow cavities, such as veins.

One or more tasks are solved by the objects of the independent claims. Advantageous further developments and preferred embodiments form the subject matter of the subclaims.

An endoscope for cardio-float applications comprises an endoscope head and a flexible sheath. A camera with a distal viewing direction is arranged in the endoscope head. The endoscope head has a shielding, earth-related, electrically conductive protective sheath, within which an image sensor and a camera driver are arranged. The endoscope head also has insulation that completely encloses at least the protective sheath and has a breakdown voltage of at least 1 kV. The flexible sheath forms the insulation.

On the one hand, the flexible sheathing ensures that the endoscope is flexible and can therefore also be inserted into blood vessels, for example, which do not run in a straight line in the body, and on the other hand that the endoscope is sufficiently electrically insulated. The electrical insulation is important over the entire length of the endoscope, as the camera is located in the endoscope head and thus at the distal end and the endoscope has electrical lines for supplying power to the camera and/or for transmitting the image data, which extend to the proximal end of the endoscope.

The design of the flexible sheathing as insulation also has the advantage that the endoscope head is already electrically insulated from the environment in the area of the endoscope head by the sheathing, so that no further or only thin further insulation needs to be provided in the endoscope head, thereby creating space for the components of the endoscope head. This enables a compact design of the endoscope head and thus the possibility of using such an endoscope to penetrate body vessels, in particular blood vessels, which were previously inaccessible to endoscopes with a camera in the endoscope head.

The electrically insulating sheath preferably extends from the endoscope head in a proximal direction at least over an area of the endoscope that can be inserted into a human or animal body, in particular from the endoscope head to a camera controller.

Unless otherwise specified, distal here means further away from a camera controller or from the connection to it. It is therefore formed from the viewpoint of the endoscope.

Proximal consequently means something that is closer to a camera controller or the connection to it.

Cardio-float applications are applications in which the tools used must fulfill the cardio-float condition. They are suitable for areas of the body that can only tolerate a very low external current without being damaged. These are, for example, the heart, the brain and/or the spinal cord. Damaged here can mean both functional impairment and partial or complete destruction of the tissue by the current flow.

The endoscope can be designed in such a way that a leakage current does not exceed 50 μA when the reference voltage, e.g. 264 V at 50 Hz, is applied.

The endoscope must not experience any malfunctions up to the level of the breakdown voltage, such as a software crash, final image failure, still image, etc. However, there may be a disturbance in the image that is visible at the time of exposure.

Because the endoscope has a flexible sheath, the endoscope can also be inserted into elongated, curved cavities. This enables different examinations and operations than with rigid endoscopes. An endoscope with a flexible sheath can thus penetrate into areas of the body that are not possible with a rigid catheter, or only with great difficulty or with a major wound. With a flexible sheath, for example, the endoscope head can also be guided through veins and arteries. The sheath can be a tube, for example, but it is also conceivable that the internal endoscope elements are wrapped or molded in.

The fact that an image sensor is arranged in the endoscope head in the distal direction means that a high-resolution image of the organ to be examined can be recorded. In contrast to systems in which an image is transmitted through the sheath to an image sensor outside the body via image conductors, a system in which the image sensor is located at the distal end of the endoscope (chip-on-tip, also known as COT) has a significantly improved resolution compared to endoscopes that work with fibers. In systems with an image sensor outside the body, where the image is transmitted via image guides, only a few pixel resolutions are possible and a surgeon can only recognize coarse structures and differences in brightness. With a camera in the body in front of the object to be examined, on the other hand, a sharp image can be viewed.

An endoscope with an image sensor is also called a videoscope or video endoscope.

The distal viewing direction of the camera here not only includes the viewing direction in a straight line parallel to the axis of the protective sheath out of the sheathing, but can also be angled up to 90°.

Image conductors are also generally not as elastic because metallic cables are more flexible. Image conductors are mostly glass conductors and are therefore also brittle and can break if subjected to too much bending force.

The earth-related, electrically conductive protective sheath ensures that any electromagnetic radiation that could be generated by the camera and its camera driver is shielded without exceeding the specified limit values and without disturbing the surroundings. The environment is, for example, the interference of other devices and systems.

The earth-related, electrically conductive protective sheath is essentially rigid and a maximum of 2 cm, preferably a maximum of 1 cm and in particular a maximum of 0.5 cm long.

A first failure mode is assumed for the approval of such endoscopes. A first failure mode is the failure of a single protective measure without consideration of further subsequent failures. The most serious first fault for such an endoscope is that the insulation is damaged. If the first fault occurs, the limitation of the leakage current still ensures that the patient is not harmed. For cardio-float applications, it is prescribed that the leakage current in the first fault case is a maximum of 50 μA.

The insulation is selected in such a way that, even if the protective sheath is live, the limit value of the leakage current is not exceeded in order to provide a patient with sufficient protection against injury.

The dielectric strength of the insulation is preferably at least 75 kV/mm, preferably at least 150 kV/mm and in particular at least 250 kV/mm.

The insulation has a wall thickness of preferably at least 0.025 mm, preferably at least 0.035 mm and preferably at least 0.050 mm.

The insulation has a wall thickness of preferably maximum 0.5 mm, preferably maximum 0.3 mm and preferably maximum 0.2 mm.

The short rigid section causes no problems when guided in the human or animal body and the rigid protective sheath provides good electrical and mechanical shielding.

The flexible sheath extends from the distal end to the proximal end and is at least 1 m, preferably at least 1.5 m and in particular at least 2.5 m long.

This means that two separate sheaths do not have to be connected to each other and the insulation also serves as insulation for the electrical cables from the endoscope head to a camera controller.

This sheath not only protects the patient electrically from the electrical cables, but also mechanically.

Preferably, the camera has an optical module that is coupled to the image sensor.

The optical module has one or more lenses to provide a sharp image of the surroundings on the sensor.

The optical module, which can have a lens arrangement and is also referred to below as the optics, allows the corresponding organs to be viewed in focus during an operation.

Preferably, the distal end region of the optical module is arranged outside the protective sheath, so that distal end regions of optical fibers are arranged adjacent to the optical module to illuminate a field of view.

The upstream optical module forms an electrical insulation at the distal end of the endoscope.

The optical fibers can be used to illuminate the dark interior of the body. The optical fibers bring light beams that are generated outside the body, for example by LEDs, to the tip of the endoscope.

In contrast to image guides, optical fibers can also be made of plastic. Furthermore, it is not so critical if a single optical fiber fails. In the event of a failure, only the illumination would be weakened, whereas if an image guide fails, the image cannot be transmitted, at least in part.

Preferably, the optical module is cast in an electrically insulating adhesive in the opening direction, which preferably connects the optical module to an inner surface of the flexible sheath.

Preferably, the adhesive is a resin, in particular an epoxy resin.

This ensures that the camera does not escape forwards and damage the patient. Resin coatings are known for their high strength and non-toxicity. For example, epoxy resin is used as an inner surface coating for food tanks and for prostheses. Furthermore, the dielectric strength of resin is very high.

Preferably, the image sensor is an area sensor with preferably at least 150×150 pixels.

The image sensor can also be an area sensor with a non-square surface and have an aspect ratio of 2:1, 3:2, 64:27, 16:9, 5:3, 14:9, 4:3, 8:5, 3:2, 4:3, or 5:4, for example.

The shorter side preferably has at least 500 pixels, preferably at least 750 pixels and preferably at least 1250 pixels.

The pixel size is at least smaller than 1.8 μm×1.8 μm, preferably at least smaller than 1.4 μm×1.4 μm and preferably at least smaller than 1.0 μm×1.0 μm, whereby the square shape is not absolutely necessary.

The image sensor preferably has an edge length of maximum 2.5 mm, 1.5 mm, 1.0 mm, preferably of maximum 0.75 mm and preferably of maximum 0.5 mm.

This provides a sufficiently sharp image to recognize organ structures.

It is particularly advantageous if the area sensor is a color sensor, which makes orientation even easier.

In the simplest case, the camera driver is a circuit board that is attached to the camera and connects it to the electrical data and supply lines.

Preferably, the camera driver is designed to perform image pre-processing.

Camera drivers convert the signals from the image sensor in such a way that they can be sent to the camera controller as information packets via the electrical cables of the endoscope.

Preferably, the protective sheath is connected to an earth-related cable. This is preferably designed with an electrically conductive plastic body at its proximal end.

This means that the protective sheath, which is itself electrically conductive, is earthed. Electrical currents can flow through this cable.

Electrically conductive plastic bodies, for example, are made of epoxy resin mixed with silver particles. This conductive epoxy resin is extremely robust and can be cast like normal plastic resin. Furthermore, plastic resin is extremely inert, i.e. it is non-toxic and does not react when it comes into contact with organic material.

In the simplest case, the earth-related cable is placed on the electrically conductive protective sheath during production and then connected with a drop of conductive plastic resin. This drop closes the entire proximal opening of the protective sheath tightly.

Preferably, the endoscope is so flexible that it can be bent into a circle with a radius of at least no more than 3 cm. The rigid protective sheath, which essentially encloses the area of the camera, is excluded here.

Particularly preferably, the endoscope is flexible in such a way that it can be bent into a circle with a radius of at least no more than 10 cm and in particular of at least no more than 5 cm. This flexibility allows the sheath to be bent to such an extent that the surgical sites intended by the surgeon, such as the heart, can be reached without having to make unnecessary incisions in the human body.

The flexibility allows the sheathing to follow the bending of veins, for example.

Preferably, the diameter of the endoscope head is a maximum of 3.5 mm, preferably a maximum of 2.0 mm and in particular a maximum of 1.2 mm.

Even if such a thin endoscope is in principle also suitable for areas of the body that allow larger diameters, such as the intestine, the endoscope presented here is designed for use in 1 narrower areas, such as certain arteries, the heart or the brain.

This very small size means that even difficult regions of the animal or human body, such as the heart, brain or spinal cord, can be reached in order to treat the patient in a minimally invasive manner.

The endoscope can therefore also be passed through smaller arteries and veins.

Preferably, interference suppression capacitors are arranged between the protective sheath and the electrical earth, which are designed in such a way that a leakage current of the endoscope head is a maximum of 50 μA, preferably a maximum of 25 μA and in particular preferably a maximum of 10 μA.

The interference suppression capacitors are electrical capacitors that discharge high-frequency interference signals to the electrical earth.

One origin of these interference signals can be caused, for example, by the endoscope itself acting as an antenna and external electrical signals then flowing out via these interference suppression capacitors.

Preferably, the interference suppression capacitors have values between 1 nF and 100 nF.

The choice of capacitors limits the leakage current to a maximum of 50 μA at a specified voltage of 230 V, for example. The capacitance of the capacitors defines the AC resistance, which has a current-limiting effect and thus limits the leakage current.

Preferably, the sheathing is at least 1 m long, preferably at least 1.5 m long and in particular at least 2.5 m long.

This makes it possible to reach the organs to be examined without having to make an incision in the body in the immediate vicinity. A body opening or a minimally invasive incision can be used that is slightly further away and less dangerous. Access to the organ to be examined can then be achieved easily through the flexible sheath.

Preferably, the breakdown voltage is at least 2 kV, preferably at least 4 kV and in particular at least 6 kV.

This ensures that there is no increased leakage current and that the patient is not harmed.

An endoscope system comprises an endoscope, as described above, and a camera controller, which has galvanically isolated electrical connections, such as power supply and data lines, to the endoscope head.

The camera controller receives the image data from the image sensor, which has been pre-processed by the camera driver. The camera controller then converts the image data so that it can be interpreted by a user. This includes, for example, conversion into a common video format, with output such as HDMI. However, it can also be designed in such a way that the image data is transmitted directly to a PC via USB, for example. Alternatively, the image can be output in analog form, e.g. via YCbCr or via standard video (SD video).

With galvanic isolation, the circuit of the endoscope and the circuit of the camera controller are not connected by electrical lines, but the information is connected via electrically non-conductive coupling elements in order to send the information from the endoscope head to the camera controller.

The operating voltage of the camera can be provided by the camera controller, whereby the voltage is galvanically isolated via DC-DC converters.

Preferably, the endoscope system has at least one working channel.

Fluids and tools can be transported into or out of the human body and/or tools can be controlled from outside the human body through a working channel. Such a working channel can be, for example, a flushing channel, a suction channel and/or a guide channel.

The flushing channel can preferably transport both liquids and gas. The substance used for flushing varies depending on the organ being examined. For example, gas, preferably nitrogen or ordinary air, is used for gastric or intestinal examinations, while a saline solution is preferably used for heart operations. Flushing ensures that other substances in the human body do not obscure the camera's view. During heart surgery, for example, the camera may be obscured by blood. This obscuring blood can then be flushed away with a saline solution.

The obscuring materials also obstruct the light emitted through the optical fibers, which illuminates the object being examined and its surroundings. The flushing channel therefore not only ensures a clear view of the camera, but also up to a certain distance in front of the endoscope head.

Preferably, the endoscope system comprises Bowden cables for controlling the endoscope system during insertion into a human or animal cavity.

A cavity is a partially delimited area that can be penetrated by an endoscope without damaging it apart from the penetration hole. Instead of a surgical penetration hole, access can also be gained via a natural body opening, e.g. nose, mouth or anus. A cavity is, for example, a blood vessel, intestine or trachea.

The Bowden cables can be used to steer the flexible sheathing in predetermined directions.

This makes it possible to guide the endoscope head through the human body, for example through veins or arteries, without the endoscope head exerting unnecessary pressure on the corresponding walls of the guide channel, such as the arteries. This allows the surgeon to guide the endoscope head to the intended location in a targeted manner.

Preferably, the Bowden cables are arranged in a catheter that is separate from the endoscope.

This allows the most suitable catheter with Bowden cables to be selected depending on the application. For example, an endoscope with a corresponding image sensor may be suitable for both heart and brain surgery, but other catheters with Bowden cables could be considered useful. The reason for this is the different working channels.

During heart surgery, for example, the catheter can be brought to the heart via the aorta.

This is not possible for spinal cord or brain surgery and another external catheter with Bowden cables could be selected as more suitable.

The invention is explained in more detail below with reference to the examples shown in the drawings. The drawings show schematically:

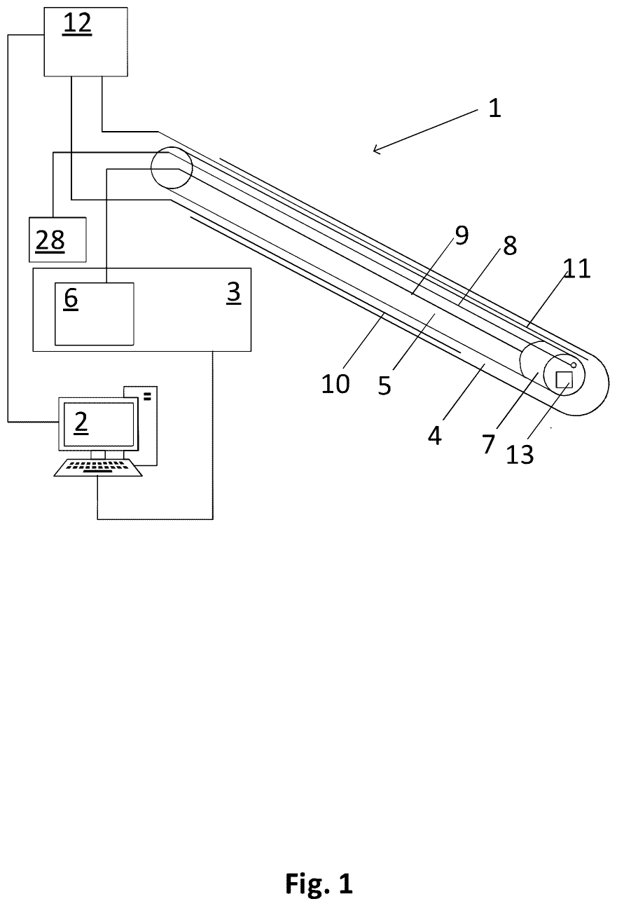

FIG. 1 An endoscope system in a block diagram,

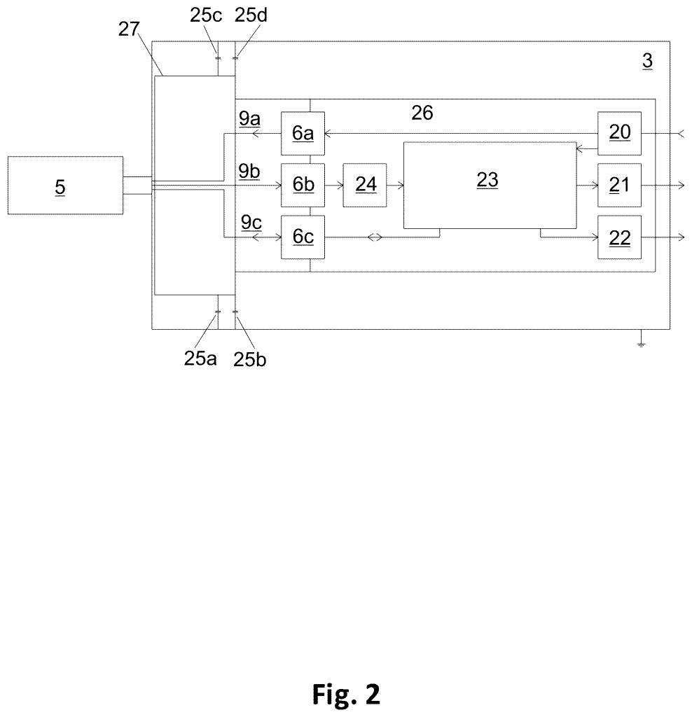

FIG. 2 a circuit diagram of a camera controller and

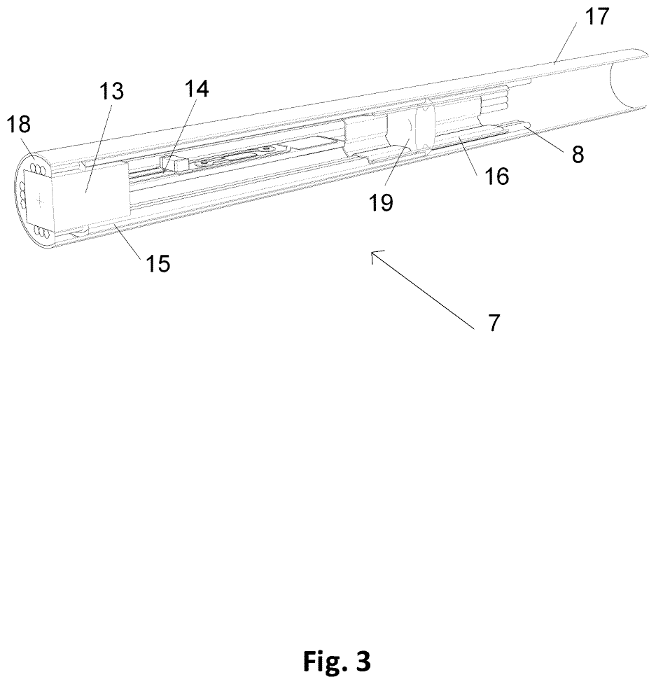

FIG. 3 a longitudinal section of an endoscope head.

An endoscope system 1 comprises an evaluation unit 2 for evaluating the image data, a camera controller 3 for processing the images, an outer catheter 4 for mechanical control of the endoscope and an endoscope 5 for imaging (FIG. 1).

The endoscope 5 is designed in such a way that it can be inserted into an outer catheter 4. Bowden cables 10 are arranged in the outer catheter 4, which can bend the catheter in predetermined directions. This enables movements in all three spatial directions.

Furthermore, four working channels 11 are provided in the outer catheter to flush the area directly in front of the distal end of the endoscope with gas or a liquid such as a saline solution.

The Bowden cables 10 and the working channels 11 are controlled via an external catheter control device 12. The outer catheter control device 12 is connected to the evaluation unit 2 via data lines. The working channels 11 also include a flushing channel.

The endoscope 5 has an endoscope head 7 in the distal direction for optical detection, optical fibers 8 for illuminating the distal surroundings of the endoscope and several electrical lines 9 for transmitting data and currents.

The optical fibers 8 and the electrical lines 9 run in a sheath 17, which is also the insulation of the endoscope head 7. In this example, the sheath 17 is 1.5 m long and has a diameter of 1.5 mm. The sheath 17 is made of polyimide.

The endoscope head 7 is arranged at the distal end of the endoscope 5 and has a camera 13, a camera driver 14, a protective sheath 15, a screen 16, an insulation 17, an epoxy seal 18 and an electrically conductive, sealing electrically conductive bond 19.

In the area behind the protective sheath the shield 16 shields the inner area from electrical radiation during sealing.

The camera driver 14 is designed as a circuit board and is located directly behind the camera 13 and both are electrically connected to each other via solder connections so that the image signals reach the camera driver 14. The camera driver 14 manages the operating voltage of the camera 13 and provides the electrical image signals from the camera 13 in a serial data stream so that they can be sent to the camera controller 3 via the electrical lines 9.

The camera 13 has an image sensor (not shown) and camera optics (not shown). It has a square cross-section.

In this example, the image sensor has an edge length of 0.7 mm. The resolution is 200×200 pixels with a pixel size of 1.75 μm×1.75 μm. The operating voltage is 3.3 V and 25 mW is required. The sensor is a color sensor and has an RGB Bayer raster.

The camera optics have a diagonal field of view of 120° and a focal length to aperture ratio of 2.8. The focal length is 0.175 mm.

The image sensor, the camera driver 14 and the connections of the electrical cables 9a-9c are located inside the electrically conductive protective sheath 15. The protective sheath 15 has a diameter of 1.3 mm. It is made of stainless steel and has a wall thickness of 0.05 mm.

The camera optics are arranged outside the protective sheath 15 in the distal direction, as a result of which the camera 13 protrudes from the protective sheath 15. The optics have a diameter of 1.3 mm.

Optical fibers 8 extend from a light source 28 through the sheath of the endoscope 5 and through the endoscope head 7 inside the protective sheath 15 to the distal end and are arranged around the camera 13. In this embodiment example, 12 optical fibers 8 are arranged in such a way that three optical fibers 8 are in contact with each side of the camera 13.

Outside the protective sheath 15 is the sheath 17, which serves as electrical insulation. It extends to the distal end of the endoscope 5 and seals with the camera optics of the camera 13 and with the optical fibers 8. It has a dielectric strength of 150 kV/mm and a wall thickness of 0.035 mm. This means that voltages of up to 5.25 kV can be applied to the protective sheath 15 without a patient suffering any damage.

At the distal tip of the endoscope, the remaining space between the camera optics and the optical fibers 8 is filled with a synthetic resin 18. On the proximal side of the endoscope head 7, the protective sheath 15 is connected to a shielding cable 27 by an electrically conductive bond 19. The electrically conductive bond 19 is an epoxy resin which is mixed with silver particles.

The length of the endoscope head 7 is 0.7 cm.

The electrical connections are electrically shielded and pass through the electrically conductive bond 19 from the camera driver 14 towards the camera controller 3.

The electrical lines 9a are formed by a two-wire power transmission with one cable to 3.3 V and one to earth (FIG. 2).

A two-wire transmission system 9b uses two cables with which a low-voltage differential signaling (LVDS signal) is transmitted. This is characterized by the fact that a low differential voltage level (maximum 5 V) is used and the signals are generated with a constant current source. The video data is transmitted via this interface.

Another two-wire connection is an I2C line 9c, which can be used to send commands to the camera driver 14. I2C is a widely used standard and is also characterized by a low voltage level.

The total of six electrical lines 9a, 9b and 9c lead through the endoscope 5 from the endoscope head 7 to the proximal end of the endoscope 5. These six electrical lines 9a, 9b and 9c have a common overall shield (not shown).

At the proximal end, the endoscope 5 has an end connector (not shown) for connecting the endoscope 5 to the camera controller 3. The end connector includes connection options for the shielding cable 27, the optical fibers 8 and the electrical lines 9.

The camera controller 3 has a camera control board 26, which is connected to the end plug of the endoscope 5 by galvanic isolators 6a-6c and has outputs on the other side for the power connection of a voltage source 20, a video output 21 for an image display unit 23 and a USB connection for a USB controller 22. The voltage source 20, the video output 21, the USB controller 22, the image display unit 23 and a deserializer 24 are arranged on this camera control board 26.

The camera controller 3 is designed in such a way that it can perform body float (BF) applications.

The voltage generated by the voltage source 20, the image information generated by the camera and the camera control signals are transmitted via galvanic isolators 6a, 6b and 6c. The galvanic isolators 6a, 6b and 6c form the insulating distance, which together with the 4.6 mm wide creepage distance is made of electrically non-conductive material. This insulating distance is arranged between the camera control board 26 and the endoscope 5.

The galvanic isolation for the power supply 6a is achieved via a so-called DC-DC coupling.

The shield 16 of the endoscope 5 is connected to the electrical ground, also known as earth, ground, PE or POAG, via interference suppression capacitors 25a, 25b, 25c and 25d. Currents that are coupled in, for example by radio waves, can flow out through these interference suppression capacitors. In this embodiment example, the interference suppression capacitors 25a-25d are arranged inside the camera controller 3. However, they can also be provided at a different location. The interference suppression capacitors 25a-25d have a capacitance of 5 nF. An electrical conductor 27, which connects the protective sheath 15 to the interference suppression capacitors 25a-25d, is arranged no closer than 2.5 mm in the air, to earth or to other electrical conductors.

The deserializer 24 converts the serial image data received from the camera driver 14 in such a way that the data can be processed by the image display unit 23.

The image display unit 23 processes the deserialized image data and generates a video data stream, which can be output to an external monitor of an evaluation unit 2 via the video interface 21.

In this embodiment, the evaluation unit 2 is a computer that can also be used to send commands to the camera controller 3 and the endoscope 5 via a USB connection connected to the USB controller 22. The evaluation unit can also communicate with the external catheter control device 12 and transmit commands.

In an alternative embodiment, the optical fibers are at least partially replaced by LEDs, which are also arranged on the endoscope head 7. These endoscope head LEDs can be arranged at or on the camera driver 14. The LEDs can also be staggered. Light is guided out distally via short optical fibers.

The LEDs can also be arranged at approximately the same height as the image sensor. Here, lenses can focus or expand the light from the LEDs.

In an alternative embodiment, the Bowden cables are connected to the endoscope.

Another possibility is that the light for the optical fibers 8 is coupled in via a separate supply. It is also possible for LEDs to be placed in the handpiece of the application part so that the end connector only has to connect the power supply to the LEDs.

In an alternative embodiment, the protective sheath can be made of a flexible braid, e.g. metal braid, or of electrically conductive plastic.

Another possibility is that the evaluation unit 2 is a screen on which the images generated by the image display unit 23 are displayed.

In an alternative embodiment, only a single optical fiber 8 is used.

### REFERENCE SIGN

1 Endoscope system
2 Evaluation unit
3 Camera controller
4 Outer catheter
5 Endoscope
6 Galvanic separation
7 Endoscope head
8 Optical fiber
9 Electrical cables
10 Bowden cables
11 Working channels
12 Outer catheter control device
13 Camera
14 Camera driver
15 Electrically conductive protective sheath
16 Screen
17 Sheathing
18 Epoxy sealant
19 Bonding
20 Voltage source
21 Video output
22 USB controller
23 Image display unit
24 Deserializer
25 Interference suppression capacitors
26 Camera control board
27 Shielding cable
28 Light generating unit

1. An endoscope for cardio-float applications comprising an endoscope head and a flexible sheath, wherein a camera with distal viewing direction is arranged in the endoscope head, wherein the endoscope head has a shielding, earth-related, electrically conductive protective sheath, within which an image sensor and a camera driver are arranged, and has an insulation which completely encloses at least the protective sheath and has a breakdown voltage of at least 1 kV, wherein the flexible sheath forms the insulation.

2. The endoscope according to claim 1, wherein the camera has an optical module which is coupled to the image sensor.

3. The endoscope according to claim 2, wherein the distal end region of the optical module is arranged outside the protective sheath, so that distal end regions of optical fibers for illuminating a field of view are arranged adjacent to the optical module.

4. The endoscope according to claim 2, wherein the optical module is surrounded in the opening direction by an electrically insulating resin layer which preferably connects the optical module to an inner surface of the flexible sheath.

5. The endoscope according to claim 1, wherein the image sensor is an area sensor with preferably at least 150×150 pixels.

6. The endoscope according to claim 1, wherein the camera driver is designed to perform image preprocessing.

7. The endoscope according to one of claim 1, wherein the protective sheath is connected at its proximal end to an electrically conductive plastic body with an earth-related cable.

8. The endoscope according to claim 1, wherein the flexible sheath is flexible such that it can be bent into a circle with a radius of at least not more than 3 cm.

9. The endoscope according to one of claim 1, wherein the diameter of the endoscope head is at most 3.5 mm, preferably at most 2.0 mm and in particular at most 1.2 mm.

10. The endoscope according to one of claim 1, wherein interference suppression capacitors are arranged between the protective sheath and the earth, which are designed in such a way that a leakage current of the endoscope head is at most 50 μA, preferably at most 25 μA and in particular preferably at most 10 μA.

11. The endoscope according to one of claim 1, wherein the sheath is at least 1 m, preferably at least 1.5 m and in particular at least 2.5 m long.

12. The endoscope according to one of claim 1, wherein the breakdown voltage is at least 2 kV, preferably at least 4 kV and in particular at least 6 kV.

13. An endoscope system comprising an endoscope according to claim 1 and a camera controller, wherein the camera controller has galvanically isolated electrical connections, including a power supply and data line, to the endoscope head.

14. The endoscope system according to claim 13, wherein the endoscope system has at least one working channel.

15. The endoscope system according to claim 13, wherein the endoscope system comprises Bowden cables for controlling the endoscope system during insertion into a human or animal cavity.

16. The endoscope system according to claim 15, wherein the Bowden cables are arranged in a catheter formed separately from the endoscope.

17. An endoscope for cardio-float applications, comprising:
an endoscope head;
a camera with distal viewing direction in the endoscope head and including an image sensor and a camera driver;
a protective sheath providing earth-related electrically conductive shielding for the endoscope head, within which the image sensor and the camera driver are arranged; and
a flexible sheath completely enclosing at least the protective sheath and having a breakdown voltage of at least 1 kV.

18. The endoscope according to claim 17, wherein that the camera has an optical module which is coupled to the image sensor.

19. The endoscope according to claim 18, wherein that the distal end region of the optical module is arranged outside the protective sheath, so that distal end regions of optical fibers for illuminating a field of view are arranged adjacent to the optical module.

20. The endoscope according to claim 18, wherein the optical module is surrounded in the opening direction by an electrically insulating resin layer which preferably connects the optical module to an inner surface of the flexible sheath.
